(19) United States
(12) Patent Application Publication
(10) Pub. No.: US 2005/0233319 A1
(43) Pub. Date: Oct. 20, 2005

(54) Methods and products for peptide-based cDNA characterization and analysis

(76) Inventor: Jonathan W. Jarvik, Pittsburgh, PA (US)

Correspondence Address: Barbara E. Johnson;WEBB ZIESENHEIM LOGSDON ORKIN & HANSON, P.C., 700 Koppers Building, 436 Seventh Avenue, Pittsburgh, PA, 15219-1818, US

(21) Appl. No.: 09/788,269

(22) Filed: Feb. 16, 2001

Related U.S. Application Data
(60) Provisional application No. 60/182,983, filed on Feb. 16, 2000.

(51) Int. Cl. C12Q 1/68; G01N 33/53

(52) U.S. Cl. 435/6; 435/7.1

(57) ABSTRACT

A method for transcript analysis in which multiple polynucleotides are derived from a multiplicity of mRNAs derived from a biological sample, with the polynucleotides having homology to at least one known reference sequence, and expressing and assessing the polypeptides. The assessment of the polypeptides focuses on at least one physical property of the polypeptides to enable determination of their sequences, sometimes with reference to one or more known reference sequences.

### RELATED APPLICATION

This application is based on priority Provisional Application Ser. No. 60/182,983, filed Feb. 16, 2000.

### FIELD OF INVENTION

This invention relates to the fields of Molecular Biology and Genetics, with particular reference to the identification and analysis of DNA molecules.

### BACKGROUND

In biology and medicine, there is frequently a need to determine the sequence of a DNA fragment. The fragment may be derived from genomic DNA of viral, procaryotic or eucaryotic origin, or it may be a derived from cDNA. In many cases, the fragment derives from a larger DNA molecule, or set of molecules, whose sequence (here defined as the reference sequence) is already known. Such cases are not rare and will become increasingly common as more and more natural DNA and cDNA sequences are deposited in available databases.

A number of methods presently exist for determining the nucleotide sequence of a DNA fragment. The most commonly applied method involves cloning the fragment in a plasmid vector of known sequence, purifying the plasmid DNA, annealing a primer complimentary to a portion of the known sequence to one strand of the molecule, extending the primer with DNA polymerase, terminating the polymerization with dideoxy nucleotides, and comparing the lengths of the various terminated molecules to reveal the nucleotide sequence 3′ to the primer. Other DNA sequencing methods exist, such as selective cleavage or sequencing by hybridization to biochips. All of these methods are based solely on in vitro DNA chemistry and biochemistry. Other well developed methods, such as SSCP (single strand conformational polymorphism analysis), heteroduplex sensitivity to nuclease analysis (EMD), and allele-specific oligonucleotide hybridization, (ASO) exist for detecting mutations or sequence polymorphisms in DNA fragments. These methods, too, are based solely on in vitro DNA chemistry and biochemistry.

Rather than examining a DNA molecule by analyzing the DNA itself, in the invention described here the DNA is incorporated into a hybrid artificial gene that is transcribed and translated to produce a hybrid peptide. Physical analysis of the peptide, in conjunction with informatic analysis of the reference sequence, allows one to identify the sequence of the DNA molecule.

The analysis of peptide size as a means to infer information about a gene goes back to at least 1965, when it was reported that phage T4 amber mutants made truncated proteins and that the size of the peptide made in an amber mutant was approximately proportional to the distance of the mutation from the 3′ end of the gene. In recent years, this phenomenon has provided the basis for the protein truncation assay for identifying nonsense and frameshift mutations in mammalian genes. In the protein truncation assay, an exon is assayed for chain termination mutations by PCR-amplifying the exon, expressing it in a cell free transcription/translation system, and examining the expressed polypeptide by SDS polyacrylamide gel electrophoresis to determine if it is smaller than a non-mutant control polypeptide. While the protein truncation assay can reveal the presence of a nonsense or frameshift mutation, it is important to note that the assay does not reveal the molecular nature or exact location of the mutation—one does not know if it is a TAG, TGA, TAA or frameshift mutation, and one only knows the approximate location of the mutation within the exon.

There presently exists well developed art by which “unknown” proteins are identified by means of coupled physical and informatic analysis. In these cases, one begins with a naturally occurring protein (or sometimes a fusion protein containing a natural amino sequences) and uses the coupled analysis to determine the protein's identity—for example, by mass spectrometric analysis of tryptic fragment masses followed by search of a database of in silico-generated tryptic fragments, in which the sequences that are the sources of the tryptic fragment data may be taken from existing protein sequence databases or may be created by in silico translation of existing nucleic acid databases. In other cases, mass analysis of peptides derived from known proteins has been used to identify sequence deviations from previously determined protein sequences.

Whereas the database search activities in the prior art (examples of which are referred to above) are aimed at protein identification and/or analysis, in the instant invention the search activity is aimed at DNA identification or analysis. Thus the two are distinctly different in concept and practice. The artificial hybrid peptides that are analyzed in the instant invention are not naturally occurring, nor are they necessarily biologically active. And yet they have distinct utility as reporters that carry information about the nucleic acids that encode them.

The analysis of peptide reporters provides a number of clear advantages over analysis of the DNA sequences that encode them. One advantage derives from the fact that a peptide is considerably smaller than the DNA that encodes it (individual amino acids averages about 110 Da each whereas the trinucleotides (triplets) that encode them average over N Daltons each. Another advantage derives from the fact that peptides are much more diverse in composition than nucleic acids, as they are composed of combinations of 20 different amino acids instead of combinations of 4 different nucleotides. Thus, by way of illustration, two random DNA fragments of identical composition (e.g., with 10 adenines, 10 thymines, 15 guanines, and 15 cytosines) are extremely unlikely to encode peptides of identical composition, and so, whereas the two nucleic acids have identical masses and cannot be distinguished on the basis of mass, the peptides that they encode will, except in statistically very rare cases, have different masses and can be readily distinguished on the basis of mass.

### SUMMARY OF THE INVENTION

In the invention described here the DNA to be analyzed is incorporated into a hybrid artificial gene that is then transcribed and translated to produce a hybrid peptide. Analysis of the peptide, rather than analysis of the DNA, is used to gain sequence data about the DNA.

Specifically, the mass and/or composition and/or partial or complete amino acid sequence of the hybrid peptide is determined, and the data are used to search for matches in data sets produced by in silico transcription and translation of hybrid artificial genes created in silico using the reference sequence, or using transformations of the reference sequence such as single nucleotide deletions or substitutions thereof. This peptide-based approach to DNA sequence-determination is fundamentally different from all other methods in the art, none of which employs transcription, translation and peptide analysis, as does the instant invention.

It is important to emphasize that the peptides that are produced and analyzed in the course of practicing the invention are not derived from naturally occurring proteins, nor did they exist anywhere prior to their production from the hybrid artificial genes. Likewise the hybrid artificial genes of the invention never existed in nature prior to their production in the course of practicing the invention.

Expected Properties of Peptides Translated from Unknown Nucleotide Sequences

The invention depends on means to translate a portion of the unknown sequence as part of a fusion peptide whose synthesis originates in the known sequence and extends into the unknown sequence that is being characterized. The unknown sequence need not comprise actual protein-coding sequence in the cell from which it originates, although it may in some cases, and so the invention is of general applicability and not confined to coding sequences. The invention also depends on means to accurately measure the mass and/or composition and/or partial or complete amino acid sequence of the fusion peptide. Many methods for making such measurements are known in the art, and a number of them will be discussed later in this specification. But first, let us consider the issue of the expected sizes, masses, and amino acid sequences of the peptides that can be translated from an unknown sequence. For the purpose of this analysis, we will make the simplifying assumption that the unknown sequence is statistically random. Later in this specification, specific examples using natural DNA sequences will be provided.

Of the 64 codons, 3 (UAA, UAG, UGA) are nonsense codons that terminate translation. Thus, in any reading frame of a random nucleotide sequence, approximately 1 of 21 codons (˜3/64) will be nonsense and approximately 20 of 21 (˜61/64) will be sense codons.

We now ask the question: if translation begins at an arbitrary nucleotide in a random DNA sequence, how large will the resulting peptide be? The answer can be given in the form of a distribution that can be calculated as follows. The likelihood that the first codon in the sequence is a nonsense codon (and that the peptide will thus be zero amino acids in length) is 1/21, or ˜4.7%. The likelihood that the first codon is not a nonsense codon and the second codon is a nonsense codon (and that the peptide will thus be one amino acid in length) is 20/21×1/21, or ˜4.5%. The likelihood that the first and second codons are not nonsense codons and the third codon is a nonsense codon (and that the peptide will thus be two amino acids in length) is 20/21×20/21×1/21, or 4.3%, and so on. Thus the likelihood that a peptide will have exactly length N is given by the expression (20/21)^{N}×1/21. Also, since the chance that a peptide will reach at least length N is (20/21)^{N}, we can readily calculate the likelihood of a peptide having a given length or less from the expression 1−(20/21)^{N}.

The table below shows the calculated probabilities, for the first 24 codons of a random DNA sequence, that a given peptide will be of a given length or less. The table indicates that, for example, 0.705 (approximately 70%) of all peptides will be 24 or fewer amino acids in length, and that 0.216 (approximately 20%) of all peptides will be 4 or fewer amino acids in length. In other words, about half of all peptides will be between 5 and 24 amino acids in length.

| Peptide | Percent of length |
|---|---|
| length (N) | N or less (1 − (20/21)^{N}) |
| 0 | 4.7 |
| 1 | 9.3 |
| 2 | 13.6 |
| 3 | 17.7 |
| 4 | 21.6 |
| 5 | 25.4 |
| 6 | 28.9 |
| 7 | 32.3 |
| 8 | 35.5 |
| 9 | 38.6 |
| 10 | 41.5 |
| 11 | 44.3 |
| 12 | 47.0 |
| 13 | 49.5 |
| 14 | 51.9 |
| 15 | 54.2 |
| 16 | 56.4 |
| 17 | 58.4 |
| 18 | 60.0 |
| 19 | 62.3 |
| 20 | 64.1 |
| 21 | 65.8 |
| 22 | 67.4 |
| 23 | 69.0 |
| 24 | 70.5 |

These expectations were tested by taking the 10,942 base pair sequence that includes the entire human nucleolin gene (Genbank accession number gb JO5584) and translating it in silico beginning at number of arbitrarily chosen positions. In particular, translation was begun at every 50th nucleotide beginning at position 2001 and ending at position 4001. The lengths of the encoded peptides, translated from the indicated position to the first in-frame nonsense codon encountered, are listed below. Those between 5 and 24 amino acids are marked with an asterisk. 17 out of the 40 peptides are between 4 and 24 amino acids in length, very close to the 20 out of 40 predicted on theoretical grounds, as described above.

| Start | Peptide length (amino acids) |
|---|---|
| 2001 | 24* |
| 2051 | 2 |
| 2101 | 21* |
| 2151 | 20* |
| 2201 | 45 |
| 2251 | 2 |
| 2301 | 20* |
| 2351 | 37 |
| 2401 | 16* |
| 2451 | 21* |
| 2501 | 25 |
| 2551 | 30 |
| 2601 | 0 |
| 2651 | 20* |
| 2701 | 13* |
| 2751 | 11* |
| 2801 | 0 |
| 2851 | 4* |
| 2901 | 21* |
| 2951 | 16* |
| 3001 | 14* |
| 3051 | 0 |
| 3101 | 69 |
| 3151 | 1 |
| 3201 | 26 |
| 3251 | 19* |
| 3301 | 211 |
| 3351 | 107 |
| 3401 | 86 |
| 3451 | 161 |
| 3501 | 79 |
| 3551 | 36 |
| 3601 | 111 |
| 3651 | 7* |
| 3701 | 42 |
| 3751 | 61 |
| 3801 | 0 |
| 3851 | 38 |
| 3901 | 11* |
| 3951 | 40 |
| 4001 | 18* |

If the nucleotide sequence is random, the probability that a sequence of a given length translated from it will have a particular amino acid sequence can be calculated simply by multiplying together the frequencies in the genetic code of the codons encoding each amino acid amino acid in the sequence. Since some amino acids have as many as six codons and others as few as one, the predicted frequency will vary depending on the amino acid sequence itself. Thus the sequence LRRLLR, made up entirely of six-codon amino acids, will appear at a frequency of 1 in (6/61)⁶, or approximately once in one million codons, and the sequence MWWMMW, made up entirely of one-codon amino acids, will appear at a frequency of 1 in (1/61)⁶, or approximately once in fifty billion codons. The frequencies of other sequences will fall between these two extremes. The important point for us is that even a relatively short sequence will appear very rarely, and so if we can determine the amino acid sequence of a peptide translated from unknown sequence, we can match it to a portion of the reference sequence with high specificity.

Let us now address the issue of the degree of specificity that can be obtained in a search of the reference sequence if we know only the mass, but not the amino acid sequence or composition, of a peptide that is translated from an unknown portion of it? For the sake of this discussion, we will assume that the mass of the peptide is determined with such accuracy as to distinguish each amino acid combination from all others. The number of distinct amino acid combinations and their frequencies is represented by the polynomial expansion (a+b+c+d+ . . . +q+r+s)^{N}, where the letters “a” through “s” (19 letters) represent the frequencies in the genetic code of each amino acid (there are 19 instead of 20 letters because two amino acids, leucine and isoleucine, have the same mass and must be treated as a group) and N represents the length of the peptide. The number of terms in the expansion represents the number of composition classes, and the value of each term divided by the sum of the values of all of the terms gives the frequency of any given class. It should be clear to the reader that for all but very small values of N, the frequency of any given class will be very low.

Depending on the size and sequence of the reference sequence, there may be just one peptide encoded in it of a given mass, or there may be more than one.

Generation of Fusion Peptides

The operation of the invention depends upon the presence of a specially engineered DNA sequence adjacent to the unknown DNA. The engineered sequence contains at minimum the following elements: (1) a promoter sequence oriented to promote transcription into the unknown sequence, and (2) a translation initiation sequence, and (3) a coding sequence comprises at minimum a start codon. Transcription from the promoter, followed by translation of the transcript beginning at the start codon, yields a fusion peptide with an N-terminal portion of known amino acid composition followed by a portion of unknown sequence encoded by the unknown DNA. A second known sequence may, in some embodiments, be incorporated into the C-terminal portion of the fusion peptide.

Analysis of Fusion Peptides

Once a fusion peptide has been produced as described above, it must be analyzed to determine its mass and/or its composition and/or its amino acid sequence. (Mass Spectrometry is one preferred analytical method because it is fast and highly accurate. A number of specific examples of the application of mass spectrometric analysis to fusion peptides are given later in this specification.) The data are compared with the data set generated in silico that contains all possible fusion peptides generated by fusing the known sequence to the reference sequence at all possible positions in the reference sequence and calculating the masses and/or compositions and/or amino acid sequences of the resulting peptides. Absence of a match, which will occur in the great majority of the positions, allows one to exclude that portion of the reference sequence from consideration, whereas a match indicates that it may indeed be the actual sequence coding for the unknown portion of the fusion peptide. If there is only one such match, and if the entire reference sequence has been scanned, then the unknown sequence has been identified. If there are multiple matches, additional data are needed to narrow the conclusion to a single site. Such data can come in a number of forms, including the generation and analysis of more than one fusion peptide from the same region the reference sequence, or the generation and analysis of peptides translated from different reading frames of the same nucleic acid sequence. Specific examples of multiple peptide analysis from nearby, adjacent or overlapping nucleotides are given below and in the claims. But it is important to state that the invention has utility even if it narrows down, but does not absolutely define, the identity of the unknown sequence.

Purification of Fusion Proteins Prior to Analysis

In some cases it may be desirable to purify the fusion peptide prior to analysis. One well established means for doing this is to include a predetermined amino acid sequence (epitope tag) in the known portion of the fusion peptide that binds to a known molecule (e.g., an antibody) or other reagent (immobilized nickel, for example). The antibody or other reagent is then used to capture and purify the peptide by immunoaffinity chromatography or immobilized metal affinity chromatography (IMAC) prior to analysis. Or a larger known sequence suitable for affinity purification such as glutathione-S-transferase (GST), thioredoxin, or maltose binding protein(MBP), may be incorporated at the N or C-terminus of the peptide. A single affinity element (tag) may be incorporated within the N or C terminal portion of the peptide, or multiple tags may be incorporated within one or both portions. When the tag is incorporated in the C-terminal portion, peptides that result from premature translation termination do not carry the tag and are not affinity purified, thereby eliminating a potential source of noise in the analysis. When different tags are incorporated within both the N and C terminal portions of the peptide, the peptide may be purified by sequential affinity capture using first one, and then the other, tag. In this case only full-length peptide is purified, eliminating potential sources of noise in the analysis due to premature translation termination, inappropriate translation initiation, or post-translational proteolysis of the peptide. Many means for separating and/or purifying peptides or proteins are also well known and may be applied in certain embodiments of the invention. These include gel electrophoresis, capillary electrophoresis, liquid chromatography (LC), capillary liquid chromatography, high performance liquid chromatography (HPLC), differential centrifugation, filtration, gel filtration, membrane chromatography, affinity purification, biomolecular interaction analysis (BIA), ligand affinity purification, glutathione-S-transferase affinity chromatography, cellulose binding protein affinity chromatography, maltose binding protein affinity chromatography, avidin/streptavidin affinity chromatography, S-tag affinity chromatography, thioredoxin affinity chromatography, metal-chelate affinity chromatography, immobilized metal affinity chromatography, epitope-tag affinity chromatography, immunoaffinity chromatography, immunoaffinity capture, capture using bioreactive mass spectrometer probes, mass spectrometric immunoassay, and immunoprecipitation.

Detection and Characterization of Mutations and DNA Polymorphisms

Certain embodiments of the invention can be used to detect and characterize naturally occurring mutations and DNA polymorphisms, including single nucleotide polymorphisms (SNPs). This is done by comparing the coding capacity of subsets of the reference sequence with the coding capacity of equivalent subsets of the sequence derived from it by specific nucleotide changes, as follows. (By coding capacity is meant the set of the amino acids encoded in at least one reading frame of a sequence; a change in the coding capacity would be due, at minimum, to a change in amino acid composition of at least one encoded peptide.) For every peptide generated in silico by translation of a sequence containing a portion of the reference sequence as described previously in this specification, an additional related set of peptides is generated by generating, also in silico, a set of transformed DNA sequences derived from the same portion of the reference DNA sequence, each member of the set containing a different sequence alteration. Each member of the transformed set is then translated in silico to give a transformed set of peptide sequences. In the case of single nucleotide substitutions, for example, since there are exactly three nucleotide changes that can be made at each position in the relevant portion of the reference DNA sequence, the expanded set of peptides will contain 3N members, where N is the length of the relevant portion of the reference nucleotide sequence. (In most cases, some of the members of the new set will be identical due to the degeneracy of the genetic code.) When the transformed data set is searched with the experimentally determined peptide data, as described previously in this specification, single nucleotide departures from the reference sequence are revealed as matches to members of the transformed data set.

In another embodiment of the invention, mutations or DNA polymorphisms are detected and quantified, by first producing a PCR amplicon representing a distinct portion of the reference sequence, such as a single exon in a gene of interest. The amplicon is expressed as part of a fusion peptide as described previously. In one embodiment, the exon is expressed in frame with respect to the translation initiation codon in the vector, with the result that the peptide comprises the entire amino acid sequence encoded in the exon. If the PCR template contains a point mutation that alters the amino acid sequence, this will be observed as, for example, a distinct change in the mass of the peptide relative to the mass of the peptide from the non-mutant exon. A large number of diseases are known to be caused by mutations in known genes, and the mutations in these genes that are responsible for dominant or recessive genetic disease may be examined using the instant invention. These include: Ataxia talangietasia (ATM), Familian adematous polypsosis (APC), Hereditary breast/ovarian cancer (BRCA1, BRCA2), Hereditary melanoma (CDK2, CDKN2), Hereditary non-polypsosis colon cancer (hMSH2, HMLH1, hPMS1, hPMS2), Hereditary retinoblastoma (RB1), Hereditary Wilm's Tumor (WT1), Li-Fraumeni syndrome (p53), Multiple endocrine neoplasia (MEN1, MEN2), Von Hippel-Lindau syndrome (VHL), Congenital adrenal hyperplasia, Androgen Receptor Mutation, Tetrahydrobiopterin deficiency, X-Linked agammaglobulinemia, Cystic Fibrosis (CFTR), Muscular Dystrophy (DMD, BMD), Factor X deficiency, Mitochondrial gene deficiency, Factor VII deficiency, Glucose-6-Phosphate deficiency, Pompe Disease, Hemophilia A, Hexosaminidase A deficiency, Human Type I and Type III Collagen deficiency X-linked SCID, Retinitis pigmentosa (RP) LIACAM deficiency, MCAD deficiency, LDL Receptor deficiency, Ornithine Transcarbamylase deficiency, PAX6 Mutation, Phenylketonuria, Tuberous Sclerosis, von Willebrand Factor Disease, Werner Syndrome.

### DESCRIPTION OF PREFERRED EMBODIMENTS

### SPECIFIC EXAMPLES

In examples 1-6 to follow, the masses of the peptides encoded in the various nucleotide sequences were calculated using the table of mass values shown below Peptide masses calculated using these values were rounded off to the nearest Dalton.

| Amino Acid | Mass | |
|---|---|---|
| Alanine | 71.0 | Da |
| Arginine | 156.1 | |
| Asparagine | 114.0 | |
| Aspartic acid | 115.0 | |
| Cysteine | 103.0 | |
| Glutamic acid | 129.0 | |
| Glutamine | 128.1 | |
| Glycine | 57.0 | |
| Histidine | 137.1 | |
| Isoleucine | 113.1 | |
| Leucine | 113.1 | |
| Lysine | 128.1 | |
| Methionine | 131.0 | |
| Phenylalanine | 147.1 | |
| Proline | 97.1 | |
| Serine | 87.0 | |
| Threonine | 101.0 | |
| Tryptophan | 186.1 | |
| Tyrosine | 163.1 | |
| Valine | 99.1 | |

### Example 1

### Identification of a Subcloned EcoRI Fragment of a Cloned Human Gene

The EMBL3 clone HG3 contains a 10942 base pair insert containing the human nucleolin gene as well as surrounding intergenic sequences (Srivistava, Genbank accession number gb JO5584). Purified HG3 DNA is digested to completion with the restriction endonuclease EcoRI and a plasmid mini-library is constructed by cloning the fragments into the EcoRI site of the vector pUC19 using standard methods. The library is transformed into competent E. coli BLR cells. Ampicillin resistant colonies are selected on LB ampicillin plates, and a single colony is picked and used to prepare a plasmid miniprep. A 250 ml liquid culture of cells from this colony is grown in LB-ampicillin medium at 25 degrees to a density of 2×10⁸ cells per ml, induced with 1 mM IPTG for 2 hours, concentrated to a volume of 10 ml by centrifugation, and lysed by sonication in the presence of the protease inhibitors AEBSF, bestatin, E-64 and pepstatin A. A second 250 ml control culture with nonrecombinant pUC19 vector is prepared in parallel. All of the above steps follow standard methods well known in the art.

A 10 μl aliquot of each cell lysate is subjected to capillary liquid chromatography (LC) followed by electrospray ionization mass spectrometry (ESI/MS) using methods and procedures well known in the art. The spectrum of the lysate from the induced cells is observed to contain a distinct peak, at a position corresponding to a mass of 5253±2 Daltons that is not observed in the control cell lysate.

To identify the nucleotide sequence responsible for the 5253 peak, the JO5584 sequence is scanned to identify each EcoRI site. 5 such sites are identified. Each EcoRI fragment is ligated, in silico, to the EcoRI site in the pUC19 vector, producing 10 possible recombinant plasmids, one for each of the two possible orientations of each insert in the vector. The predicted amino acid sequence and molecular mass of each IPTG-inducible hybrid translation product (translated from the mRNA transcribed from the lac promoter in the vector) is calculated, and the masses of the ten possible polypeptides are tabulated, as shown in the table below.

| Position of | Orientation | Predicted | |
|---|---|---|---|
| EcoRI site | in pUC19 | Peptide Mass | |
| 3190 | forward | 7070 | Daltons |
| 3190 | reverse | 5253 | |
| 4028 | forward | 3998 | |
| 4028 | reverse | 5268 | |
| 6066 | forward | 4969 | |
| 6066 | reverse | 2726 | |
| 9241 | forward | 8485 | |
| 9241 | reverse | 3109 | |
| 9543 | forward | 2840 | |
| 9543 | reverse | 3878 | |

The mass values above were computed by translating each hypothetical fusion polypeptide and removing the N-terminal methionine.

Comparison of the experimental results with the values in the table indicates reveals a match to the predicted mass value for one of the ten candidates—specifically the sequence that begins at position 3190 of the reference sequence and proceeds from right to left. Retrieval of the reference sequence beginning at position 3190 indicates that the cloned sequence begins with “GAATTCTTACACCTCATACTTTCCCAAGCCCCAACTTTCTCATCT GAAAATGGTAATAGTATCATCCTTACATGTTTAAGGTCATGAATTGCTAT GTGTA . . . (1st 100 nucleotides shown). The identification is confirmed by dideoxy sequencing from a primer 150 nucleotides upstream of the junction between the pUC19 sequence and the EcoRI fragment.

In this example the starting material was a cloned gene. If one begins instead with a cloned a cDNA library and uses identical procedures in an iterative manner, the identity of multiple members of the library are ascertained.

### Example 2

### Identification of a Subcloned EcoRI Fragment of a Cloned Human Gene Using Peptide Affinity Capture

The peptide TMITPSLHACRSTLED, representing the N-terminal 16 amino acids of the alpha-complementing factor of beta-galactosidase encoded in pUC19 (and also representing the 16 constant N-terminal amino acids in all of the peptides described in Example 1 above) is used to raise a polyclonal rabbit antibody using standard procedures.

A single ampicillin resistant E. coli colony derived from the mini-library transformation described in Example 1 is picked and induced lysates are prepared as described in Example 1. A control lysate from cells with nonrecombinant vector is prepared in parallel. Immunoreactive proteins are precipitated from the lysates by incubation of 1 ml aliquots with a 1:100 dilution of antiserum followed by precipitation with Protein-A using standard methods. The immunoprecipitate is suspended in 50 ul H₂O, and a 10 ul aliquot is suspended in 40 ul of MALDI-matrix α-cyano-4-hydroxycinnamic acid dissolved in 1:2 acetonitrile:1.5% trifluoroacetic acid (ACCA), and 100 nL applied to the MS probe, air dried, and subjected to matrix assisted laser desorption ionization time-of-flight (MALDI-TOF) mass spectrometry using methods and procedures well known in the art.

The mass spectrum of the immunoprecipitate from the induced cell lysate of the clone under examination is observed to contain a distinct peak, at a position corresponding to a mass of 8485±3 Daltons, that is not observed in the control. Comparison of the experimental results with the values in the table in example 1 above indicates that the insert begins at position 9241 of the reference sequence and proceeds from left to right in the Genbank sequence. Retrieval of the reference sequence beginning at position 9241 indicates that the cloned sequence begins with GAATTCACATAAATCGCAAATTTTTTTTTCCTTCCCAGAGCC ATCCAAAACTCTGTTTGTCAAAGGCCTGTCTGAGGATACCACTGAAGAGA CATTAAAG . . . (1st 100 nucleotides shown). The identification is confirmed by dideoxy sequencing as described in Example 1.

### Example 3

### Identification of a Subcloned EcoRI Fragment of a Cloned Human Gene: Analysis of Peptides from Multiple Reading Frames

The vector pTrip1Ex is digested with the restriction endonuclease BglII and the ends of the linearized plasmid are backfilled using Klenow fragment of E. coli DNA polymerase I. The plasmid is treated with the restriction endonuclease SmaI, blunt end ligated with DNA ligase and transformed into competent E. coli BLR cells. Ampicillin resistant colonies are selected on LB ampicillin plates, and a single colony is picked and used to prepare a plasmid miniprep. The plasmid, here named pTrip1Ex′, is linearized with EcoRI and a mini library is prepared using as inserts the set of fragments produced by complete digestion of the insert in EMBL3 human nucleolin clone described in example 1. Competent E coli TOPP-1 cells are transformed with the mini library and a single ampicillin resistant colony is isolated. A 250 ml liquid culture of cells from this colony is grown in LB-ampicillin medium at 25 degrees to a density of 2×10⁸ cells per ml, induced with 1 mM IPTG for 2 hours, concentrated to a volume of 10 ml by centrifugation, and lysed by sonication on ice with six intermittent 30 second sonication pulses. Control cells with nonrecombinant plasmid are prepared in parallel. Immunoprecipitates of both lysates are prepared as in Example 2.

An 10 ul aliquot of each immunoprecipitate is suspended in 40 ul of MALDI-matrix and subjected to MALDI-TOF mass spectrometry. The spectrum of the lysate from the plasmid-containing cells is observed to contain two distinct peaks not present in the control lysate, one at a mass of 4254±2 Daltons and the other at a msss of 2635±2 Daltons.

To identify the nucleotide sequence adjacent to the pTrip1Ex′ vector, each EcoRI site in the JO5584 sequence is identified and ligated, in silico, to the EcoRI site in the pTrip1Ex′ vector. For each such in silico construct, the amino acid sequences of the two expected hybrid translation products (from each of the start codons in the vector to the first in frame stop codons encountered in the insert) are calculated. The mass of each peptide is calculated and all 10 peptide pairs are tabulated, as shown in the table below. Comparison of the experimental results (i.e., peptides of 4255 and 2635 Da.) with the values predicted in the table indicates that the insert begins at position 4028 of the reference sequence and proceeds in the forward direction. It is concluded that the 5′ end of the sequence joined to the vector is GAATTCTCTTGGGTT TTGTGGTGTGCTAGACTTAATTACCCATGAATGATTT TGTCCTCTTGAGAAAATTTCAATAGCACATCTATTAGTGTTTTTTAT . . . (1st 100 nucleotides shown). The identification is confirmed by dideoxy sequencing from the plasmid using a primer 150 nucleotides 3‘to the pTrip1Ex’ EcoRI site.

| Position of | Orientation | Start | Predicted |
|---|---|---|---|
| EcoRI site | in pTrip1Ex′ | Codon | Peptide Mass |
| 3190 | forward | 1st | 6137 |
| 3190 | forward | 2nd | 5707 |
| 3190 | reverse | 1st | 6278 |
| 3190 | reverse | 2nd | 3891 |
| 4208 | forward | 1st | 4255 |
| 4208 | forward | 2nd | 2635 |
| 4208 | reverse | 1st | 19748 |
| 4208 | reverse | 2nd | 3905 |
| 6066 | forward | 1st | 3595 |
| 6066 | forward | 2nd | 3606 |
| 6066 | reverse | 1st | 6401 |
| 6066 | reverse | 2nd | 1363 |
| 9241 | forward | 1st | 3583 |
| 9241 | forward | 2nd | 7122 |
| 9241 | reverse | 1st | 4582 |
| 9241 | reverse | 2nd | 1746 |
| 9543 | forward | 1st | 5306 |
| 9543 | forward | 2nd | 1477 |
| 9543 | reverse | 1st | 9906 |
| 9543 | reverse | 2nd | 2516 |

The mass values above are computed by translating each hypothetical fusion polypeptide without the N-terminal methionine that is removed in vivo in E. coli.

### Example 4

### Identification of a Specific Mutation in a Human Gene

Blood is drawn from a man and wife and from their three children, an DNA is prepared from blood leukocytes of each using standard methods. Two 20-nucleotide PCR primers—one representing nucleotides 3190-3210 of the nucleolin sequence described previously (the forward primer) and the other representing the reverse complement of nucleotides 4008-4028 (the reverse primer)—are used to generate an 838 nucleotide PCR amplicon using high fidelity thermostabile proofreading DNA polymerase. The amplicon is cloned into the pTrip1Ex′ vector described previously, and 1000 transformant colonies from each amplification are pooled to create five bacterial cultures, two derived from the parents and there derived from their offspring. Each bacterial culture is treated as described in the previous example to produce five lysates and five MALDI-TOF mass spectra. The spectrum from the father shows two prominent peaks at positions corresponding to 6137 and 5707 Daltons. The same peaks are observed for the peptides derived from two of the offspring. The mother and the third child show not two peaks but three, two at 6137 and 5707 Da and a new one at 6169 Da. The new peak is 32 Da bigger than the 6137 peak, consistent with a change from valine to methionine with respect to the reference sequence. The fact that there is no new peak derived from the 5707 Da peak indicates that the base change(s) responsible for the valine-to-methionine substitution in the larger peptide is silent with respect to the reading frame encoding the 5707 Da. peptide. Of the six valine codons in the 6137 Da. peptide, only one, the GTG codon at position 3223, can be changed to give this result, the change being a G to A transition (to ATG) at position 3223. It is concluded that the mother and third child are heterozygous carriers for a single nucleotide polymorphism, a G to A transition, at position 3223. Dideoxy sequencing across the relevant region confirms this conclusion.

### Example 5

### Identification of a Specific Mutations in a Human Gene; Analysis of Pooled Samples

In this example known portions of the reference sequence are used to design PCR primers, which are then used to generate PCR products that are cloned, expressed in fusion peptides, and analyzed in a parallel fashion. The reference sequence predicts a peptide of a particular mass and composition; deviations from the prediction indicate differences in sequence from the reference sequence, in this example single nucleotide polymorphisms.

Two oligonucleotide primers are synthesized using standard methods. In one, CCCGAATTCAGCAGGTAAAAATCAAGG, the first 10 nucleotides contain an EcoRI site (underlined) and last 17 nucleotides correspond to the first 17 nucleotides of exon 2 of the human nucleolin gene. The other, GGGGAATTCTTACTCTTCTCCACTGCTAT, the last 17 nucleotides correspond to the reverse complement of the last 17 nucleotides of exon 2, followed immediately (in the sense orientation of the oligonucleotide) by the stop codon TAA and a sequence that includes an EcoRI site (underlined).

Blood is drawn from twenty individuals and PCR amplicons are produced as described in the previous example, using the two primers just described. The amplicons are pooled and cloned into the EcoRI site of pUC19 as described in example 2 above, and the bacterial cultures are treated as described in Example 2 above to produce a single MALDI-TOF mass spectrum derived from all twenty pooled samples. The spectrum shows a major peak at 6873±3 Da., corresponding the predicted mass of the fusion peptide encoded by the exon 2 reference sequence fused to the vector peptide sequence, and two smaller peaks at 6862±3 Da. and 6915±3 Da. The amplitude of the 6862 peak is approximately 1/20 of the 6872 peak, and the amplitude of the 6916 peak is approximately 1/40 that of the 6872 peak. The −10 Da. shift in the 6862 peak relative to the 6872 peak is that predicted for a single nucleotide polymorphism (SNP) that produces a proline to serine substitution in exon 2 that is already known to exist in the human population at a frequency of approximately 5%, and so it is concluded that in the 40 haploid genomes present in the twenty individuals, two copies of this polymorphism are very likely present. The +44 Da shift in the 6916 peak indicates an alanine to aspartic acid substitution in exon 2 that was not previously known, and that is present in one copy in the sample of 40 haploid genomes.

In this example the sample was heterogeneous because amplicons from a number of individual individuals were pooled prior to analysis. But the heterogeneity could, in other cases, be intrinsic to a single sample. For example, the sample could be a tumor biopsy containing, for example, a mixture of cells that are heterogeneous with respect to mutations in oncogenes or tumor suppressor genes, and so PCR amplification of the oncogene or tumor suppressor gene would yield a heterogeneous amplicon.

### Example 7

### Application of a Computer Program to Generate a Data Set of Mass Shifts for all Possible Single Nucleotide Substitutions in a Nucleotide Sequence

A computer program was written to compute the mass shifts for all single nucleotide substitutions in a nucleotide sequence. The program uses the amino acid mass values given in the table below. The input to the program is (1) a nucleotide sequence, and (2) a choice by the user of which of the six possible reading frames (3 forward and 3 reverse) to be considered. The program translates the input sequence and computes the masses of the encoded peptides. It then generates all possible single nucleotide substitutions of the sequence, computes a new set of peptides, compares them to the original peptide(s), and lists all of the mass differences between the mutant and non-mutant peptides. The program output is a listing of the peptide mass changes for all possible single nucleotide substitutions in the input sequence. The program then accepts input representing the mass-shift threshold for detection, i.e., the mass shift below which the shift is treated as not detectable. Output is a listing of all mutations in the sequence that are not detectable at the set threshold.

| Amino Acid | Symbol | Mass | |
|---|---|---|---|
| Alanine | A | 71.08 | Da |
| Arginine | R | 156.19 | |
| Asparagine | N | 114.10 | |
| Aspartic acid | D | 115.09 | |
| Cysteine | C | 103.14 | |
| Glutamic acid | E | 129.12 | |
| Glutamine | Q | 128.13 | |
| Glycine | G | 57.05 | |
| Histidine | H | 137.14 | |
| Isoleucine | I | 113.16 | |
| Leucine | L | 113.16 | |
| Lysine | K | 128.17 | |
| Methionine | M | 131.19 | |
| Phenylalanine | F | 147.18 | |
| Proline | P | 97.12 | |
| Serine | S | 87.08 | |
| Threonine | T | 101.10 | |
| Tryptophan | W | 186.21 | |
| Tyrosine | Y | 163.18 | |
| Valine | V | 99.13 | |

| | | |
|---|---|---|
| Nonsense | Z | — |

The program was run with the 24 nucleotide input sequence CAACTAGAAGAGGTAAGAAACTAT. Two reading frames were selected; the forward reading frame beginning with the first nucleotide (F1) and the reverse (antisense) reading frame beginning with the second antisense nucleotide (R2). The results are shown below.

[begin]

Enter Sequence:

| | |
|---|---|
| [input] | CAACTAGAAGAGGTAAGAAACTAT |
| [output] | Protein: QLEEVRNY |

Which reading frames would you like to examine?
1: Forward (F1)
2: Forward; first base removed (F2)
3: Forward; second base removed (F2)
4: Reverse (R1)
5: Reverse first base removed (R2)
6: Reverse second removed (R3)
   [input] 1,5

[output] MASS DIFFERENCES

| Location | Mutation | Frame F1 | Frame R2 |
|---|---|---|---|
| | None | 1032.13 | 722.89 |
| 1 | /A(K) | 0.04 | 0.00 |
| | C-{G(E) | 0.99 | 0.00 |
| | \T(Z) | −1032.13 | 0.00 |
| 2 | /G(R) | 28.06 | 0.00 |
| | (Q)A-{T(L) | −14.97 | 0.00 |
| | \C(P) | −31.01 | 0.00 |
| 3 | /G(Q) | 0.00 | 0.00 |
| | A-{T(H) | 9.01 | 0.00 |
| | \C(H) | 9.01 | 0.00 |
| 4 | /A(I) | 0.00 | 276.34 |
| | C-{G(V) | −14.03 | 276.34 |
| | \T(L) | 0.00 | 0.00 |
| 5 | /C(P) | −16.04 | 299.37 |
| | (L)T-{A(Q) | 14.97 | 226.32 |
| | \G(R) | 43.03 | 200.24 |
| 6 | /G(L) | 0.00 | 241.29 |
| | A-{T(L) | 0.00 | 241.33 |
| | \C(L) | 0.00 | 242.28 |
| 7 | /T(Z) | −790.84 | −34.02 |
| | G-{C(Q) | −0.99 | −34.02 |
| | \A(K) | −0.95 | 0.00 |
| 8 | /G(G) | −72.07 | −60.10 |
| | (E)A-{T(V) | −29.99 | 16.00 |
| | \C(A) | −58.04 | −44.04 |
| 9 | /G(E) | 0.00 | −34.02 |
| | A-{T(D) | −14.03 | −34.02 |
| | \C(D) | −14.03 | −48.05 |
| 10 | /T(Z) | −661.72 | 0.00 |
| | G-{C(Q) | −0.99 | 0.00 |
| | \A(K) | −0.95 | 0.00 |
| 11 | /G(G) | −72.07 | −16.04 |
| | (E)A-{T(V) | −29.99 | 23.98 |
| | \C(A) | −58.04 | 43.03 |
| 12 | /T(D) | −14.03 | 0.00 |
| | G-{C(D) | −14.03 | −14.03 |
| | \A(E) | 0.00 | 34.02 |
| 13 | /T(L) | 14.03 | −423.52 |
| | G-{C(L) | 14.03 | −423.52 |
| | \A(I) | 14.03 | 0.00 |
| 14 | /C(A) | −28.05 | −60.04 |
| | (V)T-{A(E) | 29.99 | −16.00 |
| | \G(G) | −42.08 | −76.10 |
| 15 | /G(V) | 0.00 | −26.04 |
| | A-{T(V) | 0.00 | −49.08 |
| | \C(V) | 0.00 | −48.09 |
| 16 | /G(G) | −99.14 | 0.00 |
| | A-{T(Z) | −433.47 | 0.00 |
| | \C(R) | 0.00 | 0.00 |
| 17 | /T(I) | −43.03 | 76.10 |
| | (R)G-{C(T) | −55.09 | 16.06 |
| | \A(K) | −28.02 | 60.10 |
| 18 | /G(R) | 0.00 | 10.04 |
| | A-{T(S) | −69.11 | 14.02 |
| | \C(S) | −69.11 | −16.00 |
| 19 | /G(D) | 0.99 | 0.00 |
| | A-{T(Y) | 49.08 | 0.00 |
| | \C(H) | 23.04 | 0.00 |
| 20 | /G(S) | −27.02 | −28.05 |
| | (N)A-{T(I) | −0.94 | 15.96 |
| | \C(T) | −13.00 | −42.08 |
| 21 | /A(K) | 14.07 | 48.05 |
| | C-{G(K) | 14.07 | 14.03 |
| | \T(N) | 0.00 | 14.03 |
| | /C(H) | −26.04 | 18.03 |
| 22 | \G(D) | −49.08 | 0.00 |
| | T-{A(N) | −48.09 | 0.00 |
| 23 | /G(C) | −60.04 | −12.06 |
| | (Y)A-{T(F) | −16.00 | 15.01 |
| | \C(S) | −76.10 | 43.03 |
| 24 | /C(Y) | 0.00 | −14.03 |
| | T-{A(Z) | −163.18 | 0.00 |
| | \G(Z) | −163.18 | 0.00 |

Enter the detection threshold:

[input] 0.8 Dalton.

[output] Undetectable amino acid substitutions: 1.(O)C-A(K)

The numbers in the first column denote each nucleotide in the sequence. Note that for each nucleotide in the input sequence there are three possible substitutions, so that the number of lines in the output data set is 72 (3×24). The amino acids encoded in each F1 codon are shown in the second column, followed by all possible single nucleotide substitutions at each position in the fourth column. The fifth column shows the amino acids encoded by the new codons, and the sixth column shows the mass change (if any) due to the amino acid substitution (if any) or translation termination (if any) due to the nucleotide substitution. The last column shows the mass changes due to the same substitutions when translation is in the R2 reading frame. The detection threshold value of 0.8 Daltons was entered; the program output indicated that only one substitution, at position 1 in the encoded peptide, would go undetected at this threshold value.

Note also that the expression of polypeptides from two reading frames makes the analysis significantly more robust than if just one reading frame is used. For example, if just reading frame 1 is used, a shift of −14.03 Daltons could be due to an E-to-D substitution at amino acid 3, or to an E-to-D substitution at amino acid 4, or to an L-to-V substitution at amino acid 2. When the additional reading frame data are considered, however, each of these possibilities is distinguished from the others and the ambiguity is thereby eliminated. Indeed, when up to six reading frames are considered, there is little or no ambiguity for the great majority of substitutions, even for sequences as long as several hundred nucleotides.

A data set/database such as that generated above can have great utility in the practice of the instant invention when searched by a computer program that searches the database using experimentally determined peptide mass data. Many such programs can be generated. One example is given below.

Enter Reference Sequence

Compute reverse complement of reference sequence

Translate beginning at each nucleotide Translate beginning at each nucleotide

Create relational database of peptides and nucleotide positions
Compute predicted masses of peptides; create relational database of peptides, masses, and nucleotide positions
   Enter experimentally determined
   mass data for peptide(s) derived from unknown sequence
Search database for correspondence between entered mass data and predicted mass values in database
   Output location of unknown sequence

### Example 8

### Analysis of Exon 2 of the Human rds/peripherin Gene

The sequence of exon 2 of the human rds/peripherin gene (Genbank accession M73531) is shown below. Intron sequence is shown in lower case; exon sequence in upper case.

gggaagcccatctccagctgtctgtttccctttaagTCGAATCAAGAGCA
ACGTGGATGGGCGGTACCTGGTGGACGGCGTCCCTTTCAGCTGCTGCAAT
CCTAGCTCGCCACGGCCCTGCATCCAGTATCAGATCACCAACAACTCAGC
ACACTACAGTTACGACCACCAGACGGAGGAGCTCAACCTGTGGGTGCGTG
GCTGCAGGGCTGCCCTGCTGAGCTACTACAGCAGCCTCATGAACTCCATG
GGTGTCGTCACGCTCCTCATTTGGCTCTTCGAGgtaggccctgggcagct
gggggtagagggtaaggagagcctcc

Two primers, of sequences GGCCCGGAATTCTCCAGCTGTCTGTTTCCCTTTAAG and AATTTACTCGAGCTACCCCCAGCTGCCCAGGGCCTAC were synthesized and used to PCR amplify rds/peripherin exon 2 from an individual known to carry a wild type allele of rds/peripherin. The amplicon was cut with EcoRI and XhoI and cloned into the EcoRI/XhoI sites of the pGEX derivative described in Nelson et al. The resulting plasmid was cut with Xho 1, treated with Klenow fragment of DNA polymerase, and self-ligated to produce a construct expected to produce a fusion protein with the sequence shown below.

MSPILGYWKIKGLVQPTRLLLEYLEEKYEEHLYERDEGDKWRNKKFELFL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLIEGRGIQDLVPHTTPHHTTPHHTTPHHTT
PQDLNSPAVCFPLSRIKSNVDGRYLVDGPFSCCNPSSPRPCIQYQITNNS
AHYSYDHQTEELNLWVRGCRAALLSYYSSLMNSMGVVTLLIWLFEVGPGQ
LGVARSSGRIVTD

The same primers were used to amplify rds/peripherin exon 2 from an individual known to carry a mutation in the exon that removes a Fini restriction site. An amplicon containing the mutation was cloned and expressed as described above for the non-mutant sequence.

Cells containing both constructs were grown to mid log phase in LB medium, induced with 1 mM IPTG, and incubated for 2 hours at 25°. Cells were collected by centrifugation and extracted with B-per according to the supplier's instructions. GST fusion proteins were purified by standard methods for analysis by MALDI-TOF mass spectrometry, which is performed as described previously.

The measured masses of the two fusion proteins are 35571±1 Da and 35630.±1 Da. The difference between the two is 59 Da, indicative of a substitution of arginine for proline in the peptide. Examination of the exon 2 sequence reveals a Fin I site (GTCCC) whose last two nucleotides are part of the first proline codon (CCT) in the sequence. It is concluded that a proline-to-arginine substitution is present at this proline. It is further concluded that the codon very likely suffered a transversion at the second position to create the arginine codon CGG. Dideoxy sequencing across the exon 2 sequence in both constructs confirms these conclusions.

### Example 9

### In Vitro Analysis of Exon 2 of Human rds/Peripherin

The amplicons described in the previous example are reamplified using the upstream primer 5′GGATCCTAATACGACTCACTATAGGGAGACCACCATGCATCACCATCAT CACCATCACCACTCTCCAGCTGTCTGTTTCCCTTTAAG and the downstream primer 5′CTTAGTCATTATACCCCCAGCTGCCCAGGGCCTAC. The upstream primer contains a T7 promoter followed by a translation initiation sequence (start codon underlined) followed by a sequence encoding eight histidines followed by sequence identical to the red/peripherin sequence immediately 5′ to rds/peripherin exon 2. The downstream primer contains two stop codons (in antisense orientation) preceding the sequence complimentary to the sequence just 3′ to red/peripherin exon 2.

The reamplification products are transcribed and translated in a coupled cell free system (transcription by T7 polymerase; translation by rabbit reticulocyte lysate) using established methods and procedures. Immobilized metal affinity chromatography is used to purify the translation products, and the translation products are analyzed by MALDI-TOF mass spectroscopy as in the previous example. The two major translation products are observed to differ by 59.1±0.8 Da, indicative of a substitution of arginine for proline in the polypeptide. By logic identical to that presented in the previous example, it is concluded that that the polypeptides differ by a proline-to-arginine substitution at the position of the first proline of the exon-encoded sequence.

Transcript Analysis

It is frequently of interest to determine the identities and quantities of a multiplicity of transcripts within a tissue or cell. Many means to make such determinations exist, including northern blotting, cDNA cloning and sequencing including EST analysis, hybridization to defined nucleic acid molecules arrayed on filters or glass (biochips) SAGE, differential display, and GeneCallin™. Embodiments of the instant invention adopted for transcript analysis have distinct advantages over the prior art in terms of throughput and dynamic range. In these embodiments, mRNA from the cell, tissue or organism of interest is first converted to cDNA using reverse transcriptase. Depending on the embodiment, reverse transcriptase may be primed by using random primers, poly-T primers, poly-T primers with one or more non-T anchoring nucleotides at their 3′ ends, or primers specific to one or a limited number of possible transcripts. After joining the cDNAs, or fragments thereof, to promoters and translation initiation sequences, the DNAs are expressed as fusion peptides, and the fusion peptides are analyzed, as described previously, to determine their mass, partial or complete amino acid composition, or partial or complete amino acid sequence. Analysis of the data with respect to a known reference sequence data set (deconvolution) reveals the identity of the transcript(s) that gave rise to the peptide(s), and, in certain further embodiments, also reveals sequence variation, for example mutations and/or sequence polymorphisms in the transcripts.

### Example 10

Poly-A+ RNA is prepared from human erythrocytes and cDNA is prepared using random hexamer primers and cloned in the vector pUC18 using standard methods. The cDNA insert from a single clone is cut with the restriction enzyme HhaI (CGC/G) and the set of fragments greater than 100 nucleotides in length are purified using a glass milk spin column and cloned into the SecII (CCGC/GG) site of a derivative of the expression vector pCITE4c(pCITE4c′) in which the BamHI site has been replaced by a SecII site. 500 of the resulting clones are pooled and plasmid DNA is prepared from the pool and used as template in an in vitro transcription/translation reaction (Novagen Single Tube), and the resulting epitope-tagged peptides are affinity purified and analyzed by MALDI-TOF mass spectrometry as described previously in this specification. The spectrum shows 4 new peaks as compared to the no-insert control.

To interpret (deconvolve) the 4 new peaks, a relational reference sequence data set is created using known transcript sequence data for human erythrocytes. This data set is created by scanning the sequence of each known erythrocyte transcript, identifying each HhaI site, generating in silico each predicted pCITE4c′ fusion peptide, and calculating the molecular mass of each predicted peptide. The data set so generated thus contains a predicted fusion protein mass signature set for each transcript. For example, and of particular relevance to the spectrum referred to above, the hemoglobin alpha 2 transcript predicts the very set of 4 mass values defined by the 4 peaks in the mass spectrum referred to above. No other erythrocyte transcript shows this pattern, and so it is concluded that the cDNA clone hemoglobin alpha 2. This conclusion is confirmed by dideoxy sequencing using a primer located in the pCITE4c′ promoter.

In the above example a single clone was analyzed. It should be apparent to the reader, however, that two or more clones can be pooled and, if the number of clones in the pool is not too great, the mass data from the pool can be deconvolved to yield the identity of each member. It should also be apparent, in view of the earlier teachings of this specification, that reference sequences can be generated that incorporate mutations of various kinds; matches of experimental data to members of these expanded data sets serves to identify mutations in the transcripts under analysis.

### Example 11

In another embodiment, the pUC18 clone described above is used as a template to generate a set of DNA molecules that, when transcribed and translated, yield a set of fusion peptides encoded in the antisense strand at the 3′ end of the transcript sequence, just before the polyA tail. This is achieved as follows.

First, the cDNA sequence is PCR amplified using a single 5′ primer in the pUC18 vector and a pool of nine anchored 3′ primers of the following composition:
5′-T7 promoter-translation initiation sequence-epitope tag-(T)₁₈-A-3′
5′-T7 promoter-transation initiation sequence-epitope tag-(T)₁₉-A-3′
5′-T7 promoter-translation initiation sequence-epitope tag-(T)₂₀-A-3′
5′T7 promoter-translation initiation sequence-epitope tag-(T)₁₈—C-3′
5′-T7 promoter-translation initiation sequence-epitope tag-(T)₁₉—C-3′
5′-T7 promoter-translation initiation sequence-epitope tag-(T)₂₀—C-3′
5′-T7 promoter-translation initiation sequence-epitope tag-(T)₁₈-G-3′
5′-T7 promoter-translation initiation sequence-epitope tag-(T)₁₉-G-3′
5′-T7 promoter-translation initiation sequence-epitope tag-(T)₂₀-G-3′

Because the primers are all anchored by non-T nucleotides at their 3′ ends, only three of them will prime a given cDNA sequence. In the case of the hemoglobin alpha 2 transcript, which ends in the sequence GCGGCAAAAAAAAAAAAAAAA . . . , the primers that are extended are those ending in G.

The PCR amplicons are transcribed and translated in vitro, and the resulting peptides are epitope-affinity captured and analyzed by MALDI-TOF MS as described previously. Three new peptide peaks are observed in the mass spectrum.

To interpret (deconvolve) the three new peaks, a relational reference sequence dataset is created using known transcript sequence data for human erythrocytes. This data set is created by scanning the sequence of each known erythrocyte transcript, identifying the polyA sites, generating, in silico, each predicted fusion peptide, and calculating the molecular masses of said peptides. The data set so generated thus contains a predicted fusion protein mass signature set for each transcript. For example, and of particular relevance to the spectrum referred to above, the hemoglobin alpha 2 transcript predicts the set of three mass values defined by the three peaks in the mass spectrum referred to above. No other erythrocyte transcript shows this pattern, and so it is concluded that the cDNA clone hemoglobin alpha 2. This conclusion is confirmed by dideoxy sequencing using a primer located in the pUC18 vector.

In the above example a single clone was analyzed. It should be apparent to the reader, however, that two or more clones can be pooled and, if the number of clones in the pool is not too great, the composite mass data from the pool can be deconvolved to yield the identity of each member. It should also be apparent, in view of the earlier teachings of this specification, that reference sequences can be generated that incorporate mutations of various kinds; matches of experimental data to members of these expanded data sets serves to identify mutations in the transcripts under analysis.

Further embodiments of the invention, additional steps are taken to further characterize the fusion peptides. An additional set of three mass peaks can be generated in the example above by, for example, cutting the PCR amplicons prior to transcription with a restriction enzyme such as CviJI, which cuts at the frequently occurring sequence rgcy, or by cutting the peptides with an enzyme such as trypsin prior to affinity capture, or after capture but prior to MALDI-TOF mass spectrometry, to yield an additional set of signature peptides. Or a different analytical technique, such as Q-TOF can be used to fragment the peptides into smaller units for signature analysis.

### Example 12

In yet another embodiment, a cDNA cloning vector is employed that permits expression of peptides representing more than one antisense reading frame without an intervening subcloning step. One such vector has the structural elements shown below.
-T7 promoter-IRES-ATG-T3 promoter-ATG-Universal Epitope-EcoRI-SmaI-

The sequence of the vector is such that the first ATG and the second ATG are in different reading frames.

cDNAs are made using 3 anchored 3′ primers of the structures [EcoRI-(T)₁₈-A], [EcoRI-(T)₁₈-G], and [EcoRI-(T)₁₈-C] and cloned into the EcoRI/SmaI sites of the vector described above. Plasmid DNA is prepared from individual clones, divided into two aliquots, and transcribed and translated in vitro using T7 polymerase for one aliquot and T3 polymerase for the other. After expression the samples are pooled, epitope affinity captured, and analyzed by MALDI-TOF mass spectrometry as described previously. The spectrum contains two new peaks, one from the peptide made from the reading frame defined by the first ATG, and the other from the reading frame defined by the second ATG. Together, the two peptides represent a mass signature characteristic of the cloned cDNA. To identify the cDNA the mass data are used to search a relational data set produced by means analogous to those described in the previous examples.

In additional embodiments, the information content of the spectra can be increased further. For example, an SP6 promoter and an associated translational start site and epitope tag can be placed in the expression vector downstream of the SmaI site, and in the antisense orientation, thereby yielding a an additional peptide for each clone.

In yet other embodiments, the complexity of the dataset that is searched can be reduced by producing multiple libraries using different anchored cDNA primers. For example, 12 sublibraries, that together represent all of the cDNAs, can be made using the following primers:
EcoRI-(T)₁₈-AT
EcoRI-(T)₁₈-AG
EcoRI-(T)₁₈-AC
EcoRI-(T)₁₈-AA
EcoRI-(T)₁₈-GT
EcoRI-(T)₁₈-GG
EcoRI-(T)₁₈-GC
EcoRI-(T)₁₈-GA
EcoRI-(T)₁₈-CT
EcoRI-(T)₁₈-CG
EcoRI-(T)₁₈-CC
EcoRI-(T)₁₈-CA

Each sublibrary is associated with a relational data set that is approximately one twelfth as complex as the data set for all twelve together, thereby reducing ambiguity in making assignments of mass signatures to individual transcripts.

In the examples given above, the only physical parameter whose value was measured was polypeptide mass. It should be clear to the reader, however, that assessing certain other polypeptide properties, such as amino acid composition or amino acid sequence, may also serve to locate an unknown sequence with respect to the reference sequence. Such data might be obtained, for example, by partial or complete digestion of the peptide, prior to spectrometry, with endopeptidases such as trypsin, chymotrypsin, or pepsin, or with aminopeptidases or carboxypeptidases. Analysis can be performed with a variety of spectrometric methods besides MALDI-TOF and ESI, such as tandem mass spectrometry (MS/MS), quadripole time of flight spectrometry (Q-TOF), or Fourier transform ion cyclotron resonance (FTICR) mass spectrometry Other analytical methods well known in the art can also be used to analyze the fusion peptides, such as gel or capillary electrophoresis or high performance liquid chromatography (HPLC). It should also be clear that the instant invention has utility even if it does not unambiguously assign an unknown sequence to just one place in the reference sequence. For example, a search might eliminate all but four positions in the reference sequence, each on a different chromosome; if the chromosomal location of the unknown sequence were known from some independent determination, such as fluorescence in situ hybridization (FISH), then the assignment could be made unambiguous. Likewise, there may be circumstances where the reference sequence is complex, representing, for example, an annotated combination of sequences derived from more than one individual, strain or species, which could be viral, procaryotic or eucaryotic. In such circumstances, the instant invention could be used, in medical, forensic or population biology contexts for example, to determine the individual, strain, or species from which the unknown DNA originated, or, conversely, it could be used to rule out an individual, strain or species as the source of origin of the unknown DNA.

Some embodiments of the invention include multiplex or pooled-sample analysis wherein peptides encoded in more than one DNA fragment are co-analyzed. For example, peptides encoded in more than one exon of a gene may be combined and analyzed in concert, or samples from multiple individuals may be pooled and analyzed together.

Some embodiments of the invention include methods for determining the sequence of a polynucleotide, comprising providing a nucleic acid fragment having homology to a known reference sequence; expressing at least one polypeptide from said fragment; and assessing at least one physical property of said at least one polypeptide to determine the sequence of said fragment by comparing said at least one property to the predicted properties of polypeptides encoded in said known reference sequence. The method also includes wherein said nucleic acid fragment contains a difference with respect to the reference sequence wherein said difference is selected from the group consisting of single nucleotide polymorphism, single nucleotide substitution, single nucleotide deletion, single nucleotide insertion, multiple nucleotide substitution, multiple nucleotide deletion, multiple nucleotide insertion, DNA duplication, DNA inversion, DNA translocation, and DNA deletion/substitution. The method further includes embodiments wherein said nucleic acid fragment comprises an exon or a cDNA. The method further includes embodiments wherein the polypeptide(s) contain heterologous epitope tags and expressed in living cells or expressed in a cell free systems such as an E. coli extract, rabbit reticulocyte extract, or wheat germ extract. The invention further includes embodiments wherein the peptides are purified by a variety of methods including gel electrophoresis, capillary electrophoresis, liquid chromatography (LC), capillary liquid chromatography, high performance liquid chromatography (HPLC), differential centrifugation, filtration, gel filtration, membrane chromatography, affinity purification, biomolecular interaction analysis (BIA), ligand affinity purification, glutathione-S-transferase affinity chromatography, cellulose binding protein affinity chromatography, maltose binding protein affinity chromatography, avidin/streptavidin affinity chromatography, S-tag affinity chromatography, thioredoxin affinity chromatography, metal-chelate affinity chromatography, immobilized metal affinity chromatography, epitope-tag affinity chromatography, immunoaffinity chromatography, immunoaffinity capture, capture using bioreactive mass spectrometer probes, mass spectrometric immunoassay, and immunoprecipitation. The method further includes embodiments wherein the physical property that is determined is mass, and wherein mass is determined by a variety of methods including mass spectrometry, MALDI-TOF mass spectrometry, electrospray ionization mass spectrometry (ESI)) tandem mass spectrometry (MS/MS), quadripole time of flight spectrometry (Q-TOF), Fourier transform ion cyclotron resonance (FTICR) mass spectrometry, gel electrophoresis, capillary electrophoresis, and high performance liquid chromatography (HPLC). The method further includes embodiments wherein the physical property that is assessed is partial or complete amino acid composition or sequence.

In another embodiment the present invention includes a method for genetic analysis comprising providing a nucleic acid fragment, expressing at least one polypeptide from the fragment, and assessing at least one physical property of said at least one polypeptide to determine the coding capacity of said fragment by comparing said at least one property to the predicted properties of polypeptides encoded in a known reference sequence. In a further embodiment the invention includes method for analyzing fragments that contain a differences with respect to the reference sequence that include of single nucleotide polymorphisms, single nucleotide substitutions, single nucleotide deletions, single nucleotide insertions, multiple nucleotide substitutions, multiple nucleotide deletions, multiple nucleotide insertions, DNA duplications, DNA inversions, DNA translocations, and DNA deletion/substitutions. In further embodiments the invention includes methods for analyzing nucleic acid fragment representing exons or cDNAs, for examining polypeptides that carry epitope tags, for examining polypeptides expressed in living cells or in cell free systems such E. coli extracts, rabbit reticulocyte extracts, and wheat germ extracts. The invention further includes embodiments wherein the peptides are purified by a variety of methods including gel electrophoresis, capillary electrophoresis, liquid chromatography (LC), capillary liquid chromatography, high performance liquid chromatography (HPLC), differential centrifugation, filtration, gel filtration, membrane chromatography, affinity purification, biomolecular interaction analysis (BIA), ligand affinity purification, glutathione-S-transferase affinity chromatography, cellulose binding protein affinity chromatography, maltose binding protein affinity chromatography, avidin/streptavidin affinity chromatography, S-tag affinity chromatography, thioredoxin affinity chromatography, metal-chelate affinity chromatography, immobilized metal affinity chromatography, epitope-tag affinity chromatography, immunoaffinity chromatography, immunoaffinity capture, capture using bioreactive mass spectrometer probes, mass spectrometric immunoassay, and immunoprecipitation. The method further includes embodiments wherein the physical property that is determined is mass, and wherein mass is determined by a variety of methods including mass spectrometry, MALDI-TOF mass spectrometry, electrospray ionization mass spectrometry (ESI)) tandem mass spectrometry (MS/MS), quadripole time of flight spectrometry (Q-TOF), Fourier transform ion cyclotron resonance (FTICR) mass spectrometry, gel electrophoresis, capillary electrophoresis, and high performance liquid chromatography (HPLC). The method further includes embodiments wherein the physical property that is assessed is partial or complete amino acid composition or sequence.

In additional embodiments, the invention includes methods for assessing a disease, condition, genotype, or phenotype comprising providing a nucleic acid fragment from a biological sample, and expressing at least one polypeptide from said fragment, and assessing at least one physical property of said at least one polypeptide to determine the sequence of said fragment by comparing said at least one property to the predicted properties of polypeptides encoded in a known reference sequence, and correlating said determined sequence with said disease, condition, genotype or phenotype. The biological sample may be obtained from a virus, organelle, cell, tissue, body part, exudate, excretion, elimination, or secretion of a healthy, diseased or deceased microorganism, protist, alga, fungus, animal or plant.

Other embodiments include diagnostic or prognostic tests for diseases, conditions, genotypes, or phenotypes comprising providing a nucleic acid fragment from a biological sample, and expressing at least one polypeptide from the fragment, and assessing at least one physical property of one or more of the polypeptides to determine the sequence of the fragment by comparing the property or properties to the predicted properties of polypeptides encoded in a known reference sequence. The sample may be obtained from a virus, organelle, cell, tissue, body part, exudate, excretion, elimination, or secretion of a healthy, diseased or deceased microorganism, protist, alga, fungus, animal or plant. In further embodiments, the test may detect heterozygote status, and it may indicate responses to drug or therapeutic treatments. The test may be for a genetic disease such as Alzheimer's disease, Ataxia talangietasia (ATM), Familial adematous polyposis (APC), Hereditary breast/ovarian cancer (BRCA1, BRCA2), Hereditary melanoma (CDK2, CDKN2), Hereditary non-polypsosis colon cancer (hMSH2, hMLH1, hPM51, hPMS2), Hereditary retinoblastoma (RB1), Hereditary Wilm's Tumor (WT1), Li-Fraumeni syndrome (p53), Multiple endocrine neoplasia (MEN1, MEN2), Von Hippel-Lindau syndrome (VHL), Congenital adrenal hyperplasia, Androgen receptor deficiency, Tetrahydrobiopterin deficiency, X-Linked agammaglobulinemia, Cystic Fibrosis (CFTR), Diabetes, Muscular Dystrophy (DMD, BMD), Factor X deficiency, Mitochondrial gene deficiency, Factor VII deficiency, Glucose-6-Phosphate deficiency, Pompe Disease, Hemophilia A, Hexosaminidase A deficiency, Human Type I and Type III Collagen deficiency X-linked SCID, Retinitis pigmentosa (RP) LIACAM deficiency, MCAD deficiency, LDL Receptor deficiency, Ornithine Transcarbamylase deficiency, PAX6 Mutation Phenylketonuria, RB1 Gene Mutation, Tuberous Sclerosis, von Willebrand Factor Disease, Werner syndrome, cancer, or an infectious disease.

Further embodiments include methods for assessing a disease, condition, genotype, or phenotype providing a nucleic acid fragment from a biological sample, and expressing at least one polypeptide from the fragment, assessing at least one physical property of one or more of the polypeptides to determine the coding capacity of the nucleic acid fragment by comparing said at least one property of the polypeptide(s) to the predicted properties of polypeptides encoded in a known reference sequence, and correlating said determined sequence with said disease, condition, genotype or phenotype. The biological sample may obtained from a virus, organelle, cell, tissue, body part, exudate, excretion, elimination, or secretion of a healthy, diseased or deceased microorganism, protist, alga, fungus, animal or plant. The particular original source may be blood, sweat, tears, urine, semen, saliva, sweat, feces, skin or hair, or it may come from the environment that the living inhabits or has inhabited, such as air, soil or water.

Further embodiments include diagnostic or prognostic tests for a disease, condition, genotype, or phenotype selecting a nucleic acid fragment taken from a virus, organelle, cell, tissue, body part, exudate, excretion, elimination, or secretion of a healthy, diseased or deceased microorganism, protist, alga, fungus, animal or plant, expressing at least one polypeptide from the fragment, assessing at least one physical property of the polypeptide(s) to determine the coding capacity of the fragment by comparing the property or properties to the predicted properties of polypeptides encoded in a known reference sequence. The particular original source of the nucleic acid may be blood, sweat, tears, urine, semen, saliva, sweat, feces, skin or hair, or it may come from the environment that the living inhabits or has inhabited, such as air, soil or water. The test may detect heterozygote status or indicate or response to a therapeutic drug or treatment. It may detect genetic disease, such Alzheimer's disease, Ataxia talangietasia (ATM), Familial adematous polyposis (APC), Hereditary breast/ovarian cancer (BRCA1, BRCA2), Hereditary melanoma (CDK2, CDKN2), Hereditary non-polypsosis colon cancer (hMSH2, hMLH1, hPMS1, hPMS2), Hereditary retinoblastoma (RB1), Hereditary Wilm's Tumor (WT1), Li-Fraumeni syndrome (p53), Multiple endocrine neoplasia (MEN1, MEN2), Von Hippel-Lindau syndrome (VHL), Congenital adrenal hyperplasia, Androgen receptor deficiency, Tetrahydrobiopterin deficiency, X-Linked agammaglobulinemia, Cystic Fibrosis (CFTR), Diabetes, Muscular Dystrophy (DMD, BMD), Factor X deficiency, Mitochondrial gene deficiency, Factor VII deficiency, Glucose-6-Phosphate deficiency, Pompe Disease, Hemophilia A, Hexosaminidase A deficiency, Human Type I and Type III Collagen deficiency X-linked SCID, Retinitis pigmentosa (RP) LIACAM deficiency, MCAD deficiency, LDL Receptor deficiency, Ornithine Transcarbamylase deficiency, PAX6 Mutation Phenylketonuria, RB1 Gene Mutation, Tuberous Sclerosis, von Willebrand Factor Disease, and Werner Syndrome, cancer, or infectious disease.

The invention further includes various polypeptides that are created in the embodiments described above.

Further embodiments of the invention take the form of data e useful for detecting and analyzing DNA mutations and polymorphisms stored in a physical medium in computer readable form a plurality of DNA sequence fragments contained within a reference DNA sequence, and the sequences of the polypeptides encoded in said DNA sequence fragments, the predicted sequences of a plurality of polypeptides encoded in a set of transformed DNA sequence fragments, each member of said set comprised of a DNA sequence related to said DNA sequence fragment by a specific change selected from the group consisting of single nucleotide polymorphism, single nucleotide substitution, single nucleotide deletion, single nucleotide insertion, multiple nucleotide substitution, multiple nucleotide deletion, multiple nucleotide insertion, DNA duplication, DNA inversion, DNA translocation, and DNA deletion/substitution; b. means for comparing the predicted sequences of said plurality of polypeptides with a test sequence to determine identity of the test sequence with a predicted sequence.

Additional embodiments include computer data structures, comprising: data storage media; and data sets in computer readable form on the data storage media representing a plurality of polypeptide fragments of polypeptides encoded by a reference polynucleotide sequence; and second data sets in computer readable form on the data storage media representing physical properties of each of the polypeptide fragments; and means for correlating empirically derived physical properties of test polypeptides with second data sets to determine the identity of the test polypeptides. The data structures may further comprising third data sets in computer readable form on said data storage media representing polynucleotide fragments encoding the polypeptide fragments; and means for correlating the identity of the test polypeptides with polynucleotide fragments represented in the third data sets. In these data the physical properties may include mass or partial or complete amino acid composition or sequence.

In yet additional embodiments, the invention includes data structures in which reference polynucleotides have a reading frame, and wherein one data set represents polypeptide fragments encoded in frame and polypeptide fragments encoded out of frame with respect to said reference polynucleotide.

Further embodiments include computer implemented methods for ascertaining the identity of nucleic acid fragments encoding polypeptides, wherein the nucleic acid fragments are fragments of known reference sequences, comprising the steps of measuring a physical property of a polypeptide comparing, in a computer, the measured physical property with a data set representing the predicted corresponding physical properties of possible polypeptides that are encoded by fragments of the reference sequence within a predetermined size range; and identifying a match between the measured physical property and a predicted physical property in the data set; and displaying or recording the results of the identifying step. The data set may includes physical properties of polypeptides encoded by in-frame and any of six out-of-frame fragments of said reference polynucleotide.

Additional embodiments of the invention include relational data sets useful for detecting and analyzing DNA mutations and polymorphisms comprising a plurality of DNA sequence fragments contained within a reference DNA sequence, the sequences of the polypeptides encoded in said DNA sequence fragments, and the predicted sequences of a plurality of polypeptides encoded in a set of transformed DNA sequence fragments, each member of said set comprised of a DNA sequence related to said DNA sequence fragment by a specific change selected from the group consisting of single nucleotide polymorphism, single nucleotide substitution, single nucleotide deletion, single nucleotide insertion, multiple nucleotide substitution, multiple nucleotide deletion, multiple nucleotide insertion, DNA duplication, DNA inversion, DNA translocation, and DNA deletion/substitution. Further embodiments include computer programs that search of these data sets.

The computer-implemented methods of the present invention can be carried out on a general purpose computer, such as, for example, a PC running the Windows, NT, Unix, or Linux operating systems, or a Macintosh personal computer. For some embodiments of the invention, a more powerful computer mainframe would be desirable. Suitable computers typically have a central processor, computer memory (such as RAM), and a storage medium, such as a floppy disk, a fixed disk or hard drive, a tape drive, an optical storage medium such as a CD, DVD, or WORM drive, a removable disk, or the like, which can store data in computer-readable form. Such computers typically have a means, such as a monitor, for displaying data or information, and are capable of storing program-generated data in RAM or in the storage medium. Such computers can also advantageously be connected to a printer, for providing a fixed record of information generated by the program.

A general purpose computer utilized in the present invention could be programmed with a specific program of the type described herein. In particular, this program would generate data sets of all possible nucleotide fragments, in all possible frames and in both orientations. It would predict and store data sets reflecting the translation products of those fragments. It would also store, in a correlatable manner, a data set reflecting a physical property (such as molecular weight) of each of those fragments. One program that could be used in the present invention would compare an empirically determined physical property of a polypeptide translated from a polynucleotide fragment from a biological sample with the data set to determine, for example, which possible polypeptide fragment or which possible polynucleotide fragment corresponds to the sample. In this manner, the identity of DNA in the sample can be determined.

In one embodiment, information directly or indirectly related to the identity of the polynucleotide fragment from the sample can be displayed, printed, and/or stored. This can include the exact identity or sequence of the polynucleotide, or a tag, label, or name associated therewith. It could also be a diagnosis of a disease, condition, genotype, or phenotype associated with that particular polynucleotide.

### CONCLUSION, RAMIFICATIONS AND SCOPE OF INVENTION

In conclusion, the invention specified here provides a novel method for analyzing cloned DNA segments and for identifying and/or assaying known or new polymorphisms or mutations in those DNA segments. The method has unique and highly useful advantages over all other methods the prior art.

The specific description of my invention presented above should not be construed as limiting its scope but rather as exemplification of certain embodiments thereof. Many other variations and applications are possible and can be practiced by one skilled in the art. For the purpose of expression, multiple promoters and translation start sites can be placed in the known sequence, on one or both sides thereof, so that the unknown sequence is translated in up to six different reading frames. Or the unknown sequence can be a PCR amplicon that is cloned into a vector in both orientations, thereby yielding a mixture of clones, some translated from one strand and some from the other. Or promoters and translation start signals can be incorporated near one or both ends of a transposable element, such as Tn3, Tn5, Tn7, Tn10, Ty, P-element, and Mariner; of a virus such as herpes virus, adenovirus, adeno-associated virus; or of a retrovirus. Fusion protein expression need not take place in bacteria, as in the examples given here, but may take place in eucaryotic cells such as yeast or mammalian cells, and cell free expression need not take place in a rabbit reticulocyte lysate, as in the example, but in other cell free systems. Other modalities for peptide capture can be used, such as incorporating biotinylated lysine in the peptides and capturing with avidin or streptavidin. Additionally, protease recognition sites may be incorporated into the known sequence to aid in fragment preparation, such as placing an enterokinase cleavage site and a poly-histidine sequence upstream of the junction to the unknown sequence so that a peptide for analysis can be released by enterokinase treatment of an affinity captured polypeptide. Further, the DNA polymorphisms that are identified and/or detected need not be limited to single nucleotide polymorphisms, as in the examples, but could be of many other kinds such as microsattelite repeats of different lengths or specific single nucleotide deletions, single nucleotide insertions, multiple nucleotide substitutions, multiple nucleotide deletions, multiple nucleotide insertions, DNA duplications, DNA inversions, DNA translocations, DNA deletion/substitutions or other chromosomal rearrangements.

A central element disclosed in this specification is a “peptide mass-signature” derived by translation of a nucleotide sequence in multiple reading frames. It should now be apparent to the reader that a characteristic peptide mass signature can be derived from any nucleic acid molecule using the methods taught in this specification. The peptide mass signature is, by itself, a distinct and classifiable derived property of any nucleic acid molecule—and as such it has unambiguous utility.

The peptide mass signature has utility in determining the sequence or coding capacity of the polynucleotide by reference to a known polynucleotide sequence, as described in numerous specific examples given in this specification. But the reader should recognize that even when the reference sequence is incompletely known, application of the operations described here can allow the reference sequence to be progressively determined, augmented and enlarged. In particular, if a polynucleotide yields a mass signature that is not predicted by the reference sequence, that polynucleotide may be sequenced by direct means such as dideoxy sequencing, and the new sequence may be added to the reference sequence database. In the extreme case, one could begin with no knowledge of the reference sequence and progressively fill it in by this approach.

It should also be apparent that using the methods disclosed in this specification, diverse nucleic acid molecules may be characterized and classified on the basis of their individual peptide mass signatures alone, since the peptide mass signature is, by itself, a distinct and classifiable derived property of each nucleic acid molecule. Thus peptide mass signatures may be used, for example, to examine the complexity of a DNA or mRNA/cDNA sample and to examine the relative concentrations of its components with no consideration given in the analysis to nucleic acid sequence. Thus the peptide mass signature itself, when obtained as taught in this specification, represents a novel and non-obvious invention with distinct utility.

1. A method for transcript analysis, comprising:
a. providing multiple polynucleotides derived from a multiplicity of mRNAs derived from a biological sample, said polynucleotides having homology to at least one known reference sequence;
b. expressing said polynucleotides; and
c. assessing at least one physical property of the expression products obtained thereby to determine the sequences of said polynucleotides by comparing said at least one physical property to the predicted properties of polypeptides encoded in said known reference sequence.

2. A method for genetic analysis, comprising:
a. providing multiple polynucleotides derived from a multiplicity of mRNAs derived from a biological sample, said polynucleotides having homology to at least one known reference sequence;
b. expressing said polynucleotides; and
c. assessing at least one physical property of the expression products obtained thereby to determine the sequences of said polynucleotides by comparing said at least one physical property to the predicted properties of polypeptides encoded in said known reference sequence.

3. A method for genetic analysis, comprising:
a. providing two or more nucleic acid samples derived from two or more biological samples, said biological samples being heterogenous and further containing multiple polynucleotides derived from a multiplicity of mRNAs derived from a biological sample;
b. expressing said polypeptides from said polynucleotides;
c. subjecting said polypeptides, in combination, to physical property assessment; and
d. comparing the results of said physical property assessment to predicted properties of polypeptides encoded in at least one reference sequence.

4. Providing a nucleic acid molecule;
expressing polypeptides from two or more reading frames of said nucleic acid sample; and
determining the masses of said polypeptides to create a peptide mass signature characteristic of said nucleic acid molecule.
